(19) United States
(12) Patent Application Publication
(10) Pub. No.: US 2018/0055022 A1
(43) Pub. Date: Mar. 1, 2018

(54) STERILE MALE INSECTS THAT SIGNIFICANTLY REDUCE LIFESPAN AND EGG-PRODUCTION OF MATED WILD-TYPE FEMALE INSECTS

(71) Applicant: The Regents of the University of California, Oakland, CA (US)

(72) Inventors: William Sullivan, Santa Cruz, CA (US); Thomas Kornberg, San Francisco, CA (US)

(73) Assignee: The Regents of the University of California, Oakland, CA (US)

(21) Appl. No.: 15/688,719

(22) Filed: Aug. 28, 2017

Related U.S. Application Data
(60) Provisional application No. 62/380,385, filed on Aug. 27, 2016.

(51) Int. Cl. A01K 67/033; C07K 14/435; C07K 14/575; C12N 15/85

(52) U.S. Cl. CPC A01K 67/0333; C07K 14/43581; C07K 14/575; C12N 15/113; A01K 2227/706; C12N 2015/8527; C12N 15/8509

(57) ABSTRACT

Provided herein are methods of generating sterile male insects. The methods include generating a insect stock comprising females with two X chromosomes each having the same centromere with male insects comprising an X chromosome and a Y chromosome each having the same centromere and mating parental male insects having an X:Y/O genome with normal females, thereby producing the sterile male insects.

### CROSS-REFERENCES TO RELATED APPLICATION

This application claims priority to U.S. Provisional Application No. 62/380,385, filed Aug. 27, 2016, which is incorporated herein by reference in its entirety.

### BACKGROUND

Pesticides have been the primary mechanism of controlling insect populations for many years but they are an imperfect remedy. They target beneficial as well as harmful insects, and they often remain in the environment years after their application and concentrate as they progress through the food chain, eventually harming apex mammalian and bird species (e.g. DDT). Moreover, as the population of the target insect declines, the efficiency of the pesticide also declines because the same amount of pesticide is required to kill fewer and fewer insects. Pesticides also have the major disadvantage that resistant strains are almost a certainty after repeated applications.

The sterile insect technique (SIT) is a method involving release of large numbers of sterile males in order to overwhelm the population of wild, fertile males. When wild females mate with one of the released sterile males, they lay a high percentage of unfertilized eggs. SIT has a number of advantages over pesticides. It targets only a single species of insects, eliminating harmful side-effects to other organisms. The main drawback of a typical SIT is technical: sterility is induced by large doses of X-irradiation that create dominant lethal mutations in mature sperm. Though effective and relatively inexpensive, X-rays also damage the genome in other cells of the adult male, and as a consequence, the sterile insects are weak, have reduced mating competitiveness and suffer early death. To compensate, vast numbers of X-irradiated sterile males must be released in order to have an effect on population, but the use of compromised males sterilized by X irradiation limit the effectiveness of SIT.

To overcome these problems, scientists have modified the insect genome with foreign modified genes. Although the effectiveness of this new generation of sterile insects remains to be determined (early indications are that they may also suffer reduced mating competitiveness and early death), releasing GMO insects into the wild is controversial. Release of GMO insects may be delayed or blocked because popular opinion, environmental, regulatory, and political issues must be navigated.

### BRIEF SUMMARY

Provided herein are methods of generating sterile male insects. The methods include mating first female insects comprising two X chromosomes each having the same centromere with first male insects comprising an X chromosome and a Y chromosome each having the same centromere, thereby producing first progeny male insects having an X:Y/O genome; and mating the first progeny male insects having an X:Y/O genome with normal females, thereby producing the sterile male insects.

### BRIEF DESCRIPTION OF THE DRAWINGS

FIG. 1 is a graph showing progeny from mating of 20 wild type Drosophila females with 20 wild type Drosophila males or the indicated number of X/O males.

FIG. 2 is a table showing the ability of X/O Drosophila males made by the disclosed method to control a population relative to irradiated X/Y Drosophila males.

FIG. 3 is a table showing the ability of X/O Drosophila males made by the disclosed method to control a population relative normal X/Y Drosophila males.

FIG. 4 is a table showing the results of an additional replication of the experiment shown in FIG. 3.

### DETAILED DESCRIPTION

Provided herein are methods of generating sterile male insects. The methods include providing first female insects comprising two X chromosomes each having the same centromere, and providing first male insects comprising an X chromosome and a Y chromosome each having the same centromere, a first mating the first female insects with the first male insects, thereby producing first progeny male insects having an X:Y/O genome, and a second mating the first progeny male insects having an X:Y/O genome with normal females, thereby producing X/O sterile male insects. Optionally, the first female insects further comprise a lethal gene operably linked to an inducible promoter on one or both of the X chromosomes. Optionally, the lethal gene is a proapoptotic gene. Optionally, the lethal gene is hid. Optionally, the lethal gene is reaper or grim. Optionally, the promoter comprises a tetracycline-controlled transactivator (tTA). Optionally, the first mating is performed in the absence of tetracycline. Optionally, the first male insects further comprise a lethal gene operably linked to an inducible promoter on the X chromosome or the Y chromosome. Optionally, the lethal gene is a proapoptotic gene. Optionally, the lethal gene is hid. Optionally, the lethal gene is reaper or grim. Optionally, the promoter is inducible at high temperature. Optionally, the second mating is performed at a high temperature. Optionally, the normal females are produced by mating females with males comprising a Y chromosome comprising a lethal gene operably linked to an inducible promoter under conditions that induce the promoter and result in expression of the lethal gene. Optionally, the insect is a Drosophila sp. Optionally, the Drosophila sp. is Drosophila melanogaster or Drosophila suzukii. Optionally, the sterile male insects have comparable viability to control male insects. Optionally, the sterile male insects are incapable of producing sperm. Optionally, the sterile male insects are not genetically modified organisms (GMOs).

Thus, disclosed herein is a method of creating large numbers of sterile male dipteran insects. The method produces males that lack a Y-chromosome but have not been traditionally genetically modified using genetic engineering techniques. The Y chromosome contains genes that have roles in male fertility but none that are needed for viability. Males lacking a Y chromosome (X/O) are as robust, mating competitive, and long-lived as normal (X/Y) males, but they lack sperm and are sterile.

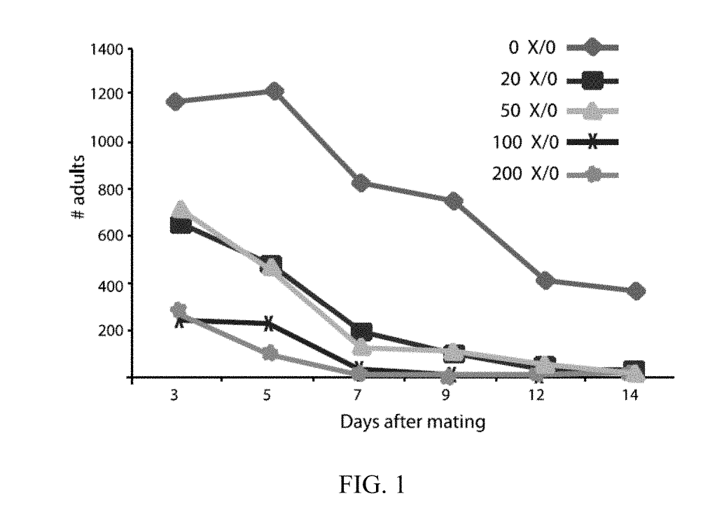

A SIT technique that releases large numbers of X/O males is more efficient and more effective than one that uses sterile males generated by X-irradiation. Using the disclosed technique, the X/O males can be produced in a single generation (12 days) using standard Mendelian genetics. Laboratory tests using Drosophila melanogaster as a model organism show that X/O males effectively suppress female fertility, even in the presence of normal males (see FIG. 1). Further, whereas <8% X-irradiated sterile males survived after two weeks and females recovered full fertility, >82% X/O males survived after two weeks and continued to suppress female fertility in the presence of normal males.

As described throughout, sterile males are generated by mating normal females or attached-X females to fertile males with a fused X and Y chromosome sharing a single centromere (X:Y/O males). As used herein, the designation X/X refers to an insect having two X chromosomes. As used herein, the designation X/Y refers to an insect having an X and a Y chromosome. The designation X:X refers to an insect with two X chromosomes that have the same centromere. The designation X:Y refers to an insect with an X and a Y chromosome, wherein the X and Y chromosomes have the same centromere. As used herein, attached-X females are female insects with two X-chromosomes with the same centromere and attached X:Y males are male insects with an X and Y chromosome with the same centromere. In a mating between X/X females and X:Y/O males, the first generation progeny will be X/X:Y (fertile females) or X/O (sterile males). Matings between X:X/0 females and X/Y males produce X/0 sterile males and X:X/Y females.

The provided methods can be applied to any insects that have a diploid heterogametic XX/XY sex determination system with males as the heterogametic sex and where X/O males are viable but sterile. Insects that have such a system include Drosophila sp., including D. melanogaster and D. suzukii.

Any attached X females, attached X:Y males, and/or normal fertile XY males can be engineered to express a gene on the attached-X or Y chromosomes that encodes an inducible lethal gene. When the lethal gene is induced, all animals carrying the lethal gene are removed from the population so that the only surviving animals in the population are sterile males. This feature advantageously does away with the need for sorting individual insects. For example, when X:X/X females are crossed to X:Y/O males, and the X:X chromosome and the X:Y chromosome carry the inducible lethal gene, then induction of expression of the gene results in lethality for all but sterile (X/O) male offspring. Similarly when X/X females are crossed to X:Y/O males, and the and the X:Y chromosome carries the inducible lethal gene, then induction of expression of the gene results in lethality for all but sterile (X/O) male offspring.

One such example of a lethal gene is the hid gene. Expression of hid (an abbreviation of head involution defective) can result in organismal death through induction of apoptosis (Heinrich J C and Scott M J, Proc Natl Acad Sci USA 97, 8229-8232 (2000); and Grether M E et al, Genes Dev 9, 1694-1708 (1995); both of which are incorporated by reference herein.) Other examples of lethal genes include reaper and grim. Methods of transformation of insect species with nucleic acids including lethal genes from the same or different insect species are known. See, e.g., U.S. Publication No. 2014/0283155.

Nucleic acid, as used herein, refers to deoxyribonucleotides or ribonucleotides and polymers and complements thereof. The term includes deoxyribonucleotides or ribonucleotides in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, conservatively modified variants of nucleic acid sequences (e.g., degenerate codon substitutions) and complementary sequences can be used in place of a particular nucleic acid sequence recited herein. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A nucleic acid is operably linked when it is placed into a functional relationship with another nucleic acid sequence. For example, DNA that encodes a presequence or secretory leader is operably linked to DNA that encodes a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are near each other, and, in the case of a secretory leader, contiguous and in reading phase. However, enhancers do not have to be contiguous. For example, a nucleic acid sequence that is operably linked to a second nucleic acid sequence is covalently linked, either directly or indirectly, to such second sequence, although any effective three-dimensional association is acceptable. A single nucleic acid sequence can be operably linked to multiple other sequences. For example, a single promoter can direct transcription of multiple RNA species. Linking can be accomplished by ligation at convenient restriction sites. If such sites do not exist, the synthetic oligonucleotide adaptors or linkers are used in accordance with conventional practice.

The terms identical or percent identity, in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be substantially identical. This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A comparison window, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981); by the homology alignment algorithm of Needleman & Wunsch, J. Mol. Biol. 48:443 (1970); by the search for similarity method of Pearson & Lipman, Proc. Nat'l. Acad. Sci. USA 85:2444 (1988); by computerized implementations of these algorithms (GAP, BESTFIT, FASTA, and TFASTA in the Wisconsin Genetics Software Package, Genetics Computer Group, 575 Science Dr., Madison, Wis.); or by manual alignment and visual inspection (see, e.g., Current Protocols in Molecular Biology (Ausubel et al., eds. 1995 supplement)).

A preferred example of an algorithm that is suitable for determining percent sequence identity and sequence similarity are the BLAST and BLAST 2.0 algorithms, which are described in Altschul et al., Nuc. Acids Res. 25:3389-3402 (1977), and Altschul et al., J. Mol. Biol. 215:403-410 (1990), respectively. BLAST and BLAST 2.0 are used, with the parameters described herein, to determine percent sequence identity for nucleic acids or proteins. Software for performing BLAST analyses is publicly available through the National Center for Biotechnology Information, as known in the art. This algorithm involves first identifying high scoring sequence pairs (HSPs) by identifying short words of a selected length (W) in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The Expectation value (E) represents the number of different alignments with scores equivalent to or better than what is expected to occur in a database search by chance. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, expectation (E) of 10, and the BLOSUM62 scoring matrix (see Henikoff & Henikoff, Proc. Natl. Acad. Sci. USA 89:10915 (1989)), alignments (B) of 50, expectation (E) of 10, M=5, N=−4, and a comparison of both strands.

The term polypeptide, as used herein, generally has its art-recognized meaning of a polymer of at least three amino acids and is intended to include peptides and proteins. However, the term is also used to refer to specific functional classes of polypeptides, such as, for example, desaturases, elongases, etc. For each such class, the present disclosure provides several examples of known sequences of such polypeptides. Those of ordinary skill in the art will appreciate, however, that the term polypeptide is intended to be sufficiently general as to encompass not only polypeptides having the complete sequence recited herein (or in a reference or database specifically mentioned herein), but also to encompass polypeptides that represent functional fragments (i.e., fragments retaining at least one activity) of such complete polypeptides. Moreover, those in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term polypeptide as used herein. Those in the art can determine other regions of similarity and/or identity by analysis of the sequences of various polypeptides described herein. As is known by those in the art, a variety of strategies are known, and tools are available, for performing comparisons of amino acid or nucleotide sequences in order to assess degrees of identity and/or similarity. These strategies include, for example, manual alignment, computer assisted sequence alignment and combinations thereof. A number of algorithms (which are generally computer implemented) for performing sequence alignment are widely available, or can be produced by one of skill in the art. Representative algorithms include, e.g., the local homology algorithm of Smith and Waterman (Adv. Appl. Math., 1981, 2: 482); the homology alignment algorithm of Needleman and Wunsch (J. Mol. Biol., 1970, 48: 443); the search for similarity method of Pearson and Lipman (Proc. Natl. Acad. Sci. (USA), 1988, 85: 2444); and/or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.). Readily available computer programs incorporating such algorithms include, for example, BLASTN, BLASTP, Gapped BLAST, PILEUP, CLUSTALW, etc. When utilizing BLAST and Gapped BLAST programs, default parameters of the respective programs may be used. Alternatively, the practitioner may use non-default parameters depending on his or her experimental and/or other requirements (see for example, the Web site having URL www.ncbi.nlm.nih.gov).

As noted above, expression of the lethal gene can be driven by an inducible promoter. An inducible promoter initiates transcription only when exposed to some particular external stimulus. Examples of such stimuli include, antibiotics such as tetracycline, hormones such as ecdysone, heavy metals, the removal of a particular amino acid or other nutrient, or application of higher than normal temperatures (heat shock promoter).

As used herein, the terms promoter, promoter element, and regulatory sequence refer to a polynucleotide that regulates expression of a selected polynucleotide sequence operably linked to the promoter, and that effects expression of the selected polynucleotide sequence in cells. Examples of promoters that are operably linked with the polynucleotide encoding one or more toxins in the lethal genetic element, include, but are not limited to, viral promoters, plant promoters and mammalian promoters. Non-limiting examples of the promoters that can be used to be operably linked with the polynucleotide encoding one or more toxins include heat shock promoters (e.g., a Hsp70 promoter (including P_Hsp70min promoter), cytomegalovirus (CMV) immediate early promoter (including human CMVmin promoter), drosophila minimal P-element promoter (Pmin), CAG promoter (which is a combination of the CMV early enhancer element and chicken beta-actin promoter, described in Alexopoulou et al. BMC Cell Biology 9:2, (2008)), simian virus 40 (SV40) promoter, the 35S RNA and 19S RNA promoters of cauliflower mosaic virus (CaMV) described in Brisson et al., Nature 1984, 310:511-514, the coat protein promoter to tobacco mosaic virus (TMV), heat shock promoters, such as soybean hsp17.5-E or hsp17.3-B described in Gurley et al., Mol. Cell. Biol. 1986, 6:559-565, and any variants thereof.

The phrase selection agent, as used herein refers to an agent that introduces a selective pressure on an organism either in favor of or against the organism that bears an inducible lethal gene. For example, the selection agent is an antibiotic, e.g., tetracycline. Optionally, the selection agent is heat.

The term transformed, as used in reference to insects, refers to insects that have undergone transformation as described herein such that the insects carry exogenous or heterologous genetic material (e.g., a recombinant nucleic acid).

The term introduce, as used herein with reference to introduction of a nucleic acid into a cell or organism, is intended to have its broadest meaning and to encompass introduction, for example by transformation methods (e.g., calcium-chloride-mediated transformation, electroporation, particle bombardment), and also introduction by other methods including transduction, conjugation, and mating. Optionally, a construct is utilized to introduce a nucleic acid into a cell or organism.

The methods, systems and components described herein are applicable to various types of insects. For example, the insect can be a direct pest or indirect pest. As used herein, “direct pests” refers to insects that can cause damage at one or more stage of their life cycle by, for example, eating crops or damaging animals. The spotted wing Drosophila, Drosophila suzukii is an important pest of many fruit crops and Drosophila pulchrella, like D. suzukii oviposits in healthy, whole fruit rather than damaged or overripe fruit (Walsh D B et al, J Integrated Pest Management 2, G1-G7 (2011); incorporated by reference herein).

Also provided is a method of biological control comprising releasing into an environment a population of sterile male insects produced using the provided methods and allowing the sterile male insects to mate with female insects in the environment, wherein the number of viable progeny in the next generation is reduced compared to release of a control insect into the environment. Optionally, the sterile male insects shorten the lifespan of the females to which they mate. Optionally, the sterile male insects suppress egg production of the female insects to which they mate. Optionally, the sterile male insects have comparable viability to control male insects.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

The examples below are intended to further illustrate certain aspects of the methods and compositions described herein, and are not intended to limit the scope of the claims.

### EXAMPLES

### Example 1

### Generation of Sterile Insects

The model organism Drosophila melanogaster was used because the Drosophila sp. Y chromosome contains genes that have roles in male fertility but none that are needed for viability. Drosophila males lacking a Y chromosome (X/O) are as robust, mating competitive, and long-lived as normal (X/Y) males, but they lack sperm and are sterile. A SIT technique that releases large numbers of X/O males should be more efficient and more effective than one that uses sterile males generated by X-irradiation. The added benefit is that large numbers of X/O males can be produced efficiently and inexpensively in a single generation (12 days) using standard Mendelian genetics. The data show that X/O males effectively suppress female fertility, even in the presence of normal males (see FIG. 1). The data also show <8% X-irradiated sterile males survived after two weeks and females recovered full fertility while >82% X/O males survived and continued to suppress female fertility in the presence of normal males.

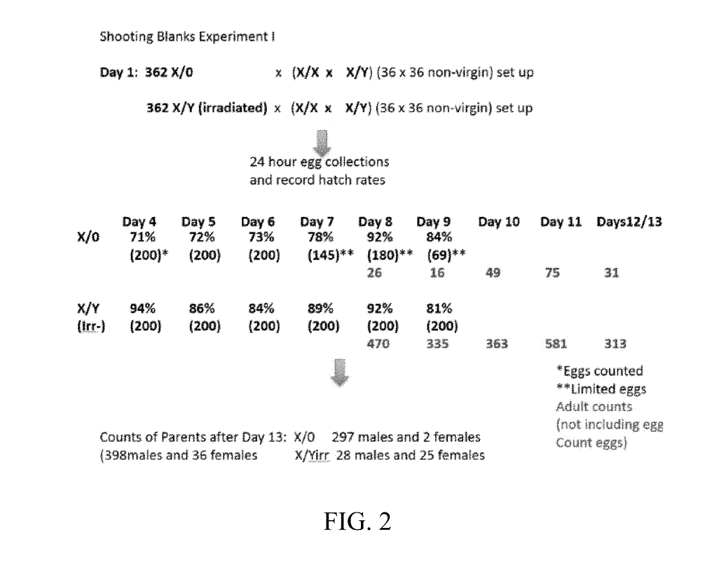

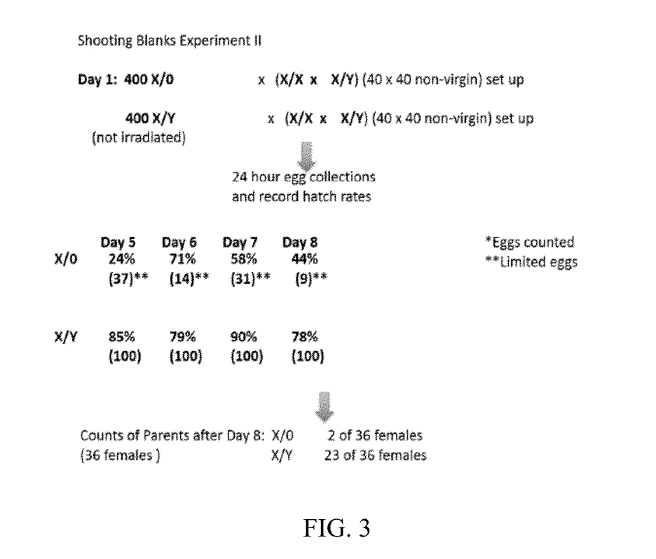

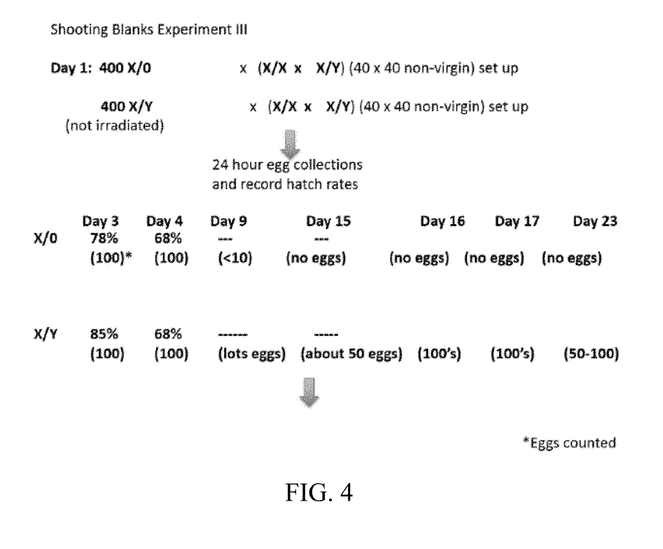

To generate X/O sterile males, parental stocks are provided such that in a single generation large numbers of sterile male insects were produced. Specifically, parental stocks with the chromosomes X:X/O (females), X:Y/O (males) and X/Y males carrying a lethal gene operably linked to an inducible promoter, in this case hid linked to a heat shock promoter, were produced. A tetracycline inducible hid transgene was present on one of the X chromosomes of the X:X/O females such that hid was only expressed in the absence of tetracycline referred to as X:X[tet-hid]. A heat shock inducible hid transgene was present on the X chromosome of the X:Y/O males so that hid is expressed only a high temperature referred to as X[hs-hid]:Y. The parental X:X[tet-hid] females and X[hs-hid]:Y males are raised in the absence of tetracycline producing only X[hs-hid]:Y male progeny. Mating normal females with X/Y[hs-hid] (males carrying heat shock inducible lethal gene on the Y chromosome) in the presence of heat produces only females. Mating these females with the X[hs-hid]:Y males in the presence of heat produces only sterile X/O males. These X/O males were then crossed with normal females to determine whether the sterile males can reduce insect populations. FIGS. 2, 3, and 4 show the results of these experiments. The results in FIGS. 2, 3, and 4, show (i) X/O males survive much better than X-irradiated males, (ii) in X/O crosses egg laying by wild-type females is greatly reduced after 6 to 9 days and (iii) in X/O crosses survival of wild type females is greatly reduced.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of generating sterile male insects comprising
providing first female insects comprising two X chromosomes each having the same centromere;
providing first male insects comprising an X chromosome and a Y chromosome each having the same centromere;
performing a first mating between the first female insects with the first male insects, thereby producing first progeny male insects having an X:Y/O genome; and
performing a second mating between the first progeny male insects having an X:Y/O genome with normal females comprising two X chromosomes that do not have the same centromere, thereby producing the X/O sterile male insects.

2. The method of claim 1, wherein the first female insects further comprise a lethal gene operably linked to an inducible promoter on one or both of the X chromosomes.

3. The method of claim 2, wherein the lethal gene is a proapoptotic gene.

4. The method of claim 2, wherein the lethal gene is hid.

5. The method of claim 2, wherein the promoter comprises a tetracycline-controlled transactivator (tTA).

6. The method of claim 5, wherein the first mating is performed in the absence of tetracycline.

7. The method of claim 1, wherein the first male insects further comprise a lethal gene operably linked to an inducible promoter on the X chromosome or the Y chromosome.

8. The method of claim 7, wherein the lethal gene is a proapoptotic gene.

9. The method of claim 7, wherein the lethal gene is hid.

10. The method of claim 7, wherein the promoter is inducible at high temperature.

11. The method of claim 10, wherein the second mating is performed at a high temperature.

12. The method of claim 1, wherein the normal females are produced by mating females with males comprising a Y chromosome comprising a lethal gene operably linked to an inducible promoter under conditions that induce the promoter and result in expression of the lethal gene.

13. The method of claim 1, wherein the insect is a Drosophila sp.

14. The method of claim 1, wherein the insect is Drosophila suzukii or Drosophila pulchrella.

15. The method of claim 1, wherein the sterile male insects have comparable viability to control male insects.

16. The method of claim 1, wherein the sterile male insects are incapable of producing sperm.

17. A method of biological control comprising:
(a) releasing into an environment a population of sterile male insects produced by:
(i) providing first female insects comprising two X chromosomes each having the same centromere;
(ii) providing first male insects comprising an X chromosome and a Y chromosome each having the same centromere;
(iii) performing a first mating between the first female insects with the first male insects, thereby producing first progeny male insects having an X:Y/O genome; and
(iv) performing a second mating between the first progeny male insects having an X:Y/O genome with normal females comprising two X chromosomes that do not have the same centromere, thereby producing the X/O sterile male insects, and
(b) allowing the sterile male insects to mate with female insects in the environment, wherein the number of viable progeny in the next generation is reduced compared to release of a control insect into the environment.

18. The method of claim 17, wherein the sterile male insects shorten the lifespan of the females to which they mate.

19. The method of claim 17, wherein the sterile male insects suppress egg production of the female insects to which they mate.

20. The method of claim 17, wherein the sterile male insects have comparable viability to control male insects.

21. The method of claim 17, wherein the sterile male insects are Drosophila suzukii or Drosophila pulchrella.
